(19) United States
(12) Patent Application Publication
(10) Pub. No.: US 2011/0160839 A1
(43) Pub. Date: Jun. 30, 2011

(54) Endoprosthesis

(75) Inventors: Jan Weber, Maastricht (NL); Liliana Atanasoska, Edina, MN (US); Rajesh Radhakrishnan, Maple Grove, MN (US)

(73) Assignee: BOSTON SCIENTIFIC SCIMED, INC., Maple Grove, MN (US)

(21) Appl. No.: 12/649,007

(22) Filed: Dec. 29, 2009

(51) Int. Cl. A61F 2/82; B05D 3/00

(52) U.S. Cl. 623/1.15; 623/1.46; 623/1.42; 427/2.24

(57) ABSTRACT

An endoprosthesis, e.g., a bioerodable stent, that is treated by plasma immersion ion implantation on the surface and optionally in the bulk. Releasable therapeutic agent, drug, or pharmaceutically active compound may be incorporated into the treated surface of the endoprosthesis to provide desired medical benefits.

### TECHNICAL FIELD

The present invention relates to endoprostheses, and more particularly to stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

It is sometimes desirable for an implanted endoprosthesis to erode over time within the passageway. For example, a fully erodable endoprosthesis does not remain as a permanent object in the body, which may help the passageway recover to its natural condition. Erodible endoprostheses can be formed from, e.g., a polymeric material, such as polylactic acid, or from a metallic material such as magnesium, iron or an alloy thereof.

### SUMMARY

The present invention is directed to an endoprosthesis, such as, for example, a stent, that is treated by plasma immersion ion implantation.

At least a portion of the surface of the endoprosthesis is treated by plasma immersion ion implantation. This treatment can provide an increased or enhanced surface area over a portion of the endoprosthesis. In an implementation the dissolution rate of a bioerodible stent can be controlled by the enhanced surface area formed by plasma immersion ion implantation over a portion of the surface of the stent.

At least a portion of the bulk of the endoprosthesis can also be treated by plasma immersion ion implantation. In some aspects, a plurality of portions of the bulk of the endoprosthesis are treated to different chemical compositions. For example, layers of different chemical compositions that have different erosion rates can be created, using plasma immersion ion implantation, at different depths in the bulk of the endoprosthesis to achieve a desired erosion sequence. In the present invention, the bulk modification and the surface modification can be advantageously achieved in a single plasma immersion ion implantation process.

After the plasma immersion ion implantation treatment, a coating can be deposited over the treated surface of the endoprosthesis to provide a desired function. Examples of suitable coatings include a tie layer, a biocompatible coating, a radiopaque metal or alloy, a drug-eluting layer, or a combination thereof.

At least one releasable therapeutic agent, drug, or pharmaceutically active compound can be incorporated into the treated surface of the endoprosthesis to provide various medical benefits. Examples of suitable therapeutic agents, drugs, or pharmaceutically active compounds include anti-thrombogenic agents, antioxidants, anti-inflammatory agents, anesthetic agents, anti-coagulants, antibiotics, and combinations thereof. In the present invention, the therapeutic agent, drug, or pharmaceutically active compound can be directly incorporated into the pores generated by the plasma immersion ion implantation treatment on the surface of the endoprosthesis, thereby eliminating the need for using carrier coatings.

The endoprosthesis may comprise a bioerodable material, e.g., a bioerodable metal or a bioerodable polymer. Examples of bioerodable metals include iron, magnesium, and an alloy thereof. Examples of bioerodable polymers include polydioxanone, polycaprolactone, polygluconate, polylactic acid-polyethylene oxide copolymer, modified cellulose, collagen, poly(hydroxybutyrate), polyanhydride, polyphosphoester, poly(amino acid), poly-L-lactide, poly-D-lactide, polyglycolide, poly(alpha-hydroxy acid), and combination thereof.

The endoprosthesis may also comprise a non-bioerodable material. Examples of suitable non-bioerodable include stainless steels, platinum enhanced stainless steels, cobalt-chromium alloys, nickel-titanium alloys, and combinations thereof.

In the present invention, the endoprosthesis can have any desired shape and size, can be self-expandable or balloon-expandable, can have any suitable transverse cross-section, and can be configured for both vascular and non-vascular lumens.

Aspects, implementations or embodiments can have one or more of the following advantages. The endoprosthesis may not need to be removed from a lumen after implantation. The endoprosthesis can have a low thrombogenecity and high initial strength. The endoprosthesis can exhibit reduced spring back (recoil) after expansion. Lumens implanted with the endoprostheses can exhibit reduced restenosis. The rate of erosion or dissolution of the endoprostheses can be controlled. The rate of erosion or dissolution of different portions of the endoprosthesis can be controlled allowing the endoprosthesis to erode in a predetermined manner, reducing the likelihood of uncontrolled fragmentation. For example, a predetermined manner of erosion can be at a first relatively slow rate, and then at a second relatively fast rate. Or the manner of erosion can be different over different portions of the stent, e.g., slower around critical structural members such as radial bands or connecting members. The manner of erosion can be from an inside of the endoprosthesis to an outside of the endoprosthesis, from an outside of the endoprosthesis to an inside of the endoprosthesis, or from a first portion to a second portion of the endoprosthesis.

The details of one or more embodiments are set forth in the accompanying drawings and the description below. Other features, objects, and advantages will be apparent from the description and drawings, and from the claims.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of an embodiment of a stent.

FIG. 3 is a perspective view of an embodiment of a stent.

FIGS. 4A-B are micrograph depictions of a region of enhanced surface-area morphology on a stent.

FIG. 5A depicts a stent having erosion enhancing regions on connectors between bands.

FIG. 5B depicts a stent after the erosion of the connectors between bands.

FIGS. 6A-C depict various circumferential cross-sectional views of a stent member.

FIGS. 7A-C depict various longitudinal cross-sectional views of a stent.

FIG. 8 depicts a plasma immersion ion implantation system.

FIG. 9 is a chart showing the range of depth at which ions can be implanted.

### DETAILED DESCRIPTION

A bioerodible endoprosthesis includes at least a portion of a surface having an enhanced or increased surface area. It has been found that treatment of endoprostheses by plasma immersion ion implantation (“PIII”) results in a surface area at the treated location that is beneficial to the controlled dissolution of the bioerodible material. Moreover, treatment of an endoprosthesis with plasma immersion ion implantation can effect or change the chemical composition of the bulk material below the surface of the endoprosthesis, thereby facilitating further control of the dissolution of the endoprosthesis. In aspects disclosed herein, an endoprosthesis treated with plasma immersion ion implantation can include a surface region having an enhanced or increased surface area. In further aspects disclosed herein, an endoprosthesis treated with plasma immersion ion implantation can include one or more layers or regions within the bulk material of the endoprosthesis having varying chemical compositions.

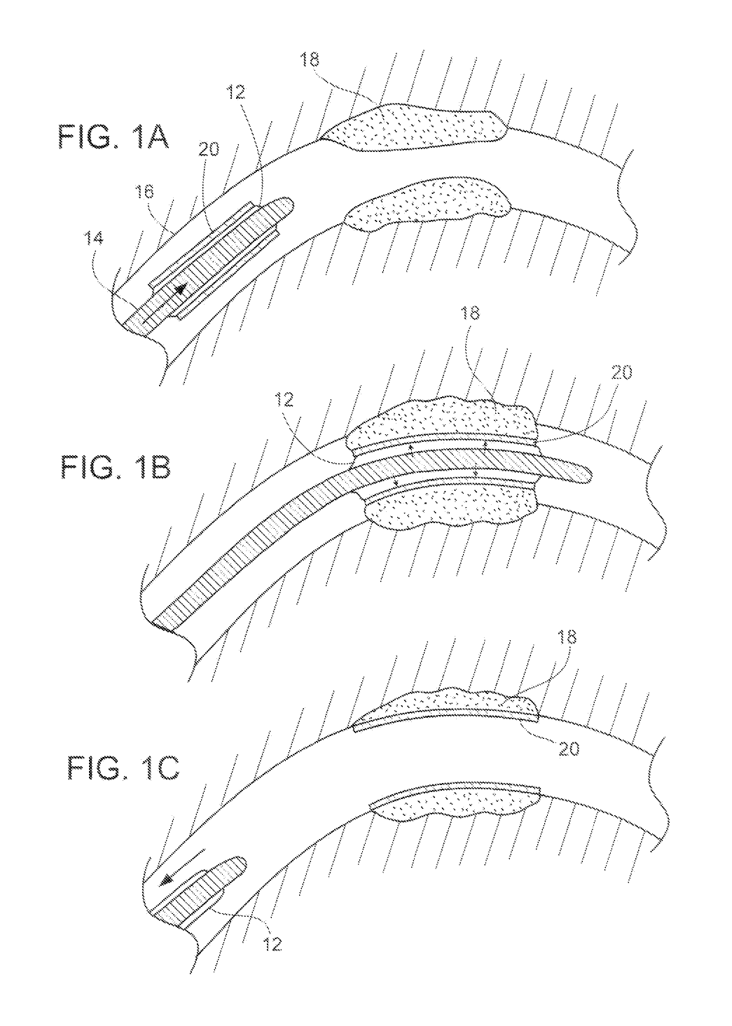

Endoprostheses can included stents, stent-grafts, grafts and filters. In an implementation, and referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded, e.g. by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

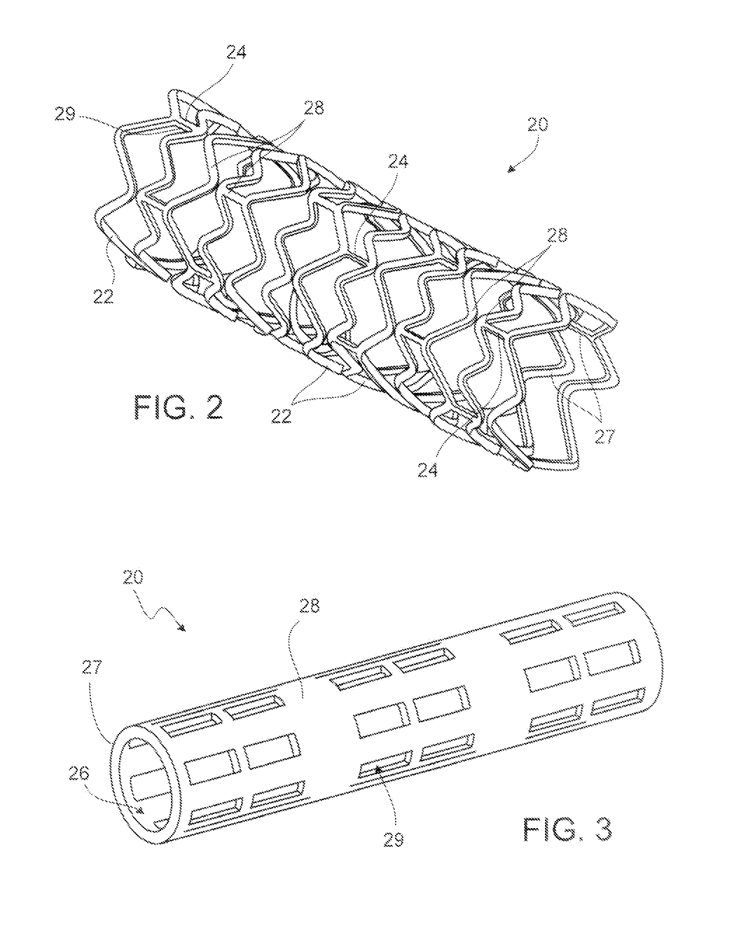

Referring to FIG. 2, an expandable stent 20 can have a stent body having the form of a tubular member defined by a plurality of bands 22 and a plurality of connectors 24 that extend between and connect adjacent bands. During use, bands 22 can be expanded from an initial, smaller diameter to a larger diameter to contact stent 20 against a wall of a vessel, thereby maintaining the patency of the vessel. Connectors 24 can provide stent 20 with flexibility and conformability that allow the stent to adapt to the contours of the vessel. Stent body 20, bands 22 and connectors 24 can have a luminal surface 26, an abluminal surface 28, and a sidewall surface 29.

Stent 20 can include a bioerodable material, e.g., a bioerodable metal or a bioerodable polymer. A bioerodable metal can be a substantially pure metallic element or an alloy. Examples of bioerodable metallic elements include iron and magnesium. Examples of bioerodable alloys include iron alloys having, by weight, 88-99.8% iron and less than 5% of other elements (e.g., magnesium and/or zinc); or 90-96% iron plus 0-5% other metals. Examples of bioerodable alloys also include magnesium alloys having, by weight, 50-98% magnesium, 0-40% lithium, 0-5% iron and less than 5% other metals or rare earths; or 79-97% magnesium, 2-5% aluminum, 0-12% lithium and 1-4% rare earths (such as cerium, lanthanum, neodymium and/or praseodymium); or 85-91% magnesium, 6-12% lithium, 2% aluminum and 1% rare earths; or 86-97% magnesium, 0-8% lithium, 2-4% aluminum and 1-2% rare earths; or 8.5-9.5% aluminum, 0.15%-0.4% manganese, 0.45-0.9% zinc and the remainder magnesium; or 4.5-5.3% aluminum, 0.28%-0.5% manganese and the remainder magnesium; or 55-65% magnesium, 30-40% lithium and 0-5% other metals and/or rare earths. Bioerodable magnesium alloys are also available under the names AZ91D, AM50A, and AE42. Other bioerodable alloys are described in Bolz, U.S. Pat. No. 6,287,332 (e.g., zinc-titanium alloy and sodium-magnesium alloys); Heublein, U.S. Patent Application 2002000406; and Park, Science and Technology of Advanced Materials, 2, 73-78 (2001), the entire disclosure of each of which is herein incorporated by reference. In particular, Park describes Mg—X—Ca alloys, e.g., Mg—Al—Si—Ca, Mg—Zn—Ca alloys. Examples of bioerodable polymers include polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), and combinations thereof.

Stent 20 can also include a non-bioerodable material. Examples of suitable non-bioerodable materials include stainless steels, platinum enhanced stainless steels, cobalt-chromium alloys, nickel-titanium alloys, and combinations thereof. In some embodiments, stent 20 can include bioerodable and non-bioerodable portions.

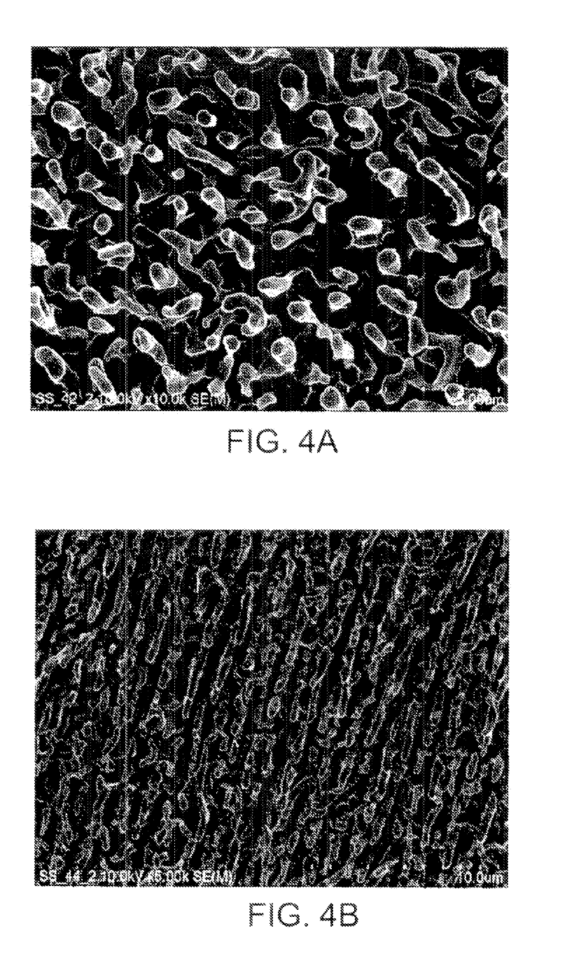

Referring to FIG. 3, the stent 20 defines a flow passage 25 there through and is capable of maintaining patency in a blood vessel. The stent 20 can include a body 27 including a surface 28. The stent body 27 can include iron or an alloy thereof. In some embodiments, the stent 20 can include a body 27 including one or more bioerodible metals, such as magnesium, zinc, iron, or alloys thereof. The body 27 can include bioerodible and non-bioerodible materials. The body 27 can have a surface including bioerodible metals, polymeric materials, or ceramics. The body 27 can have a surface 28 including an oxide of a bioerodible metal.

The stent body 27 can have a surface 28 having a morphology characterized by high-surface-area structures. Surface 28 can be on the abluminal, luminal or sidewall surfaces of stent 20. At least a portion of the stent body 27 can be treated by plasma immersion ion implantation, described further below, in order to increase the surface area of portions of surface 28 or to provide a region of surface 28 having an enhanced-surface-area morphology, such as additional surface roughness or porosity.

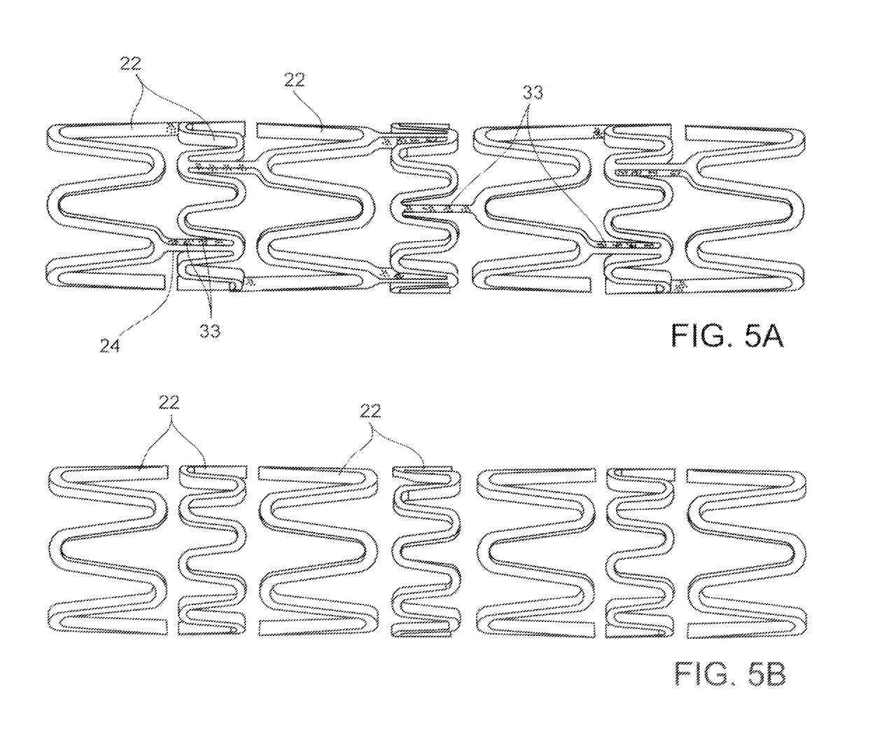

Regions of enhanced-surface-area morphology are depicted in FIGS. 4A and 4B, which, for purposes of illustration, are scanning electron micrographs taken of a cross-section of a sample of stainless steal at 1,500× and 10,000× magnifications respectively. Similar enhanced-surface-area morphology can be expected in samples of iron. The sample has been treated in a plasma immersion ion implantation processed as described herein. Scales are provided on the lower left portion of each micrograph. The micrographs show depressed surface portions as light areas and raised surface portions as dark areas. In plasma immersion ion implantation processes with Argon on an iron surface, the surface features can have a dimension of between 1-3 micron in depth and width.

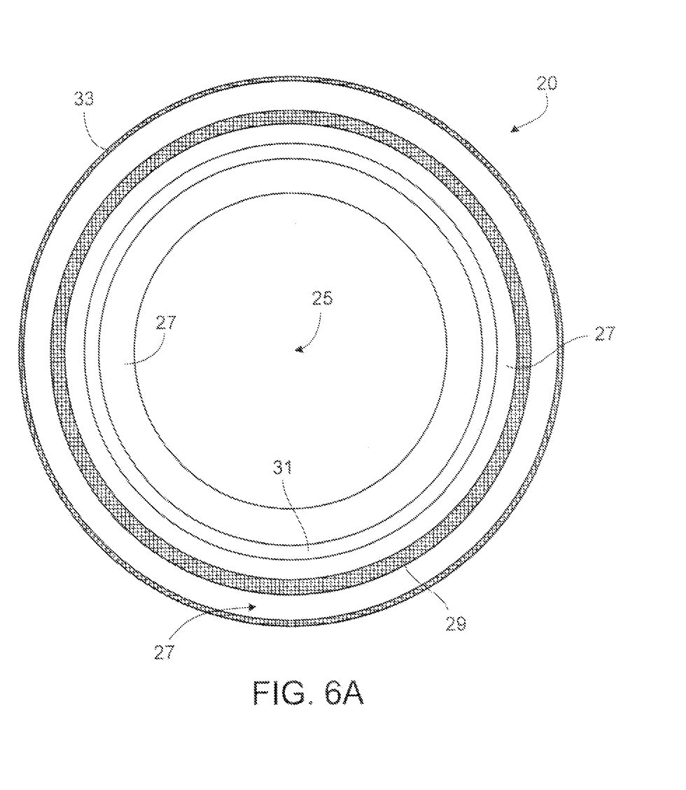

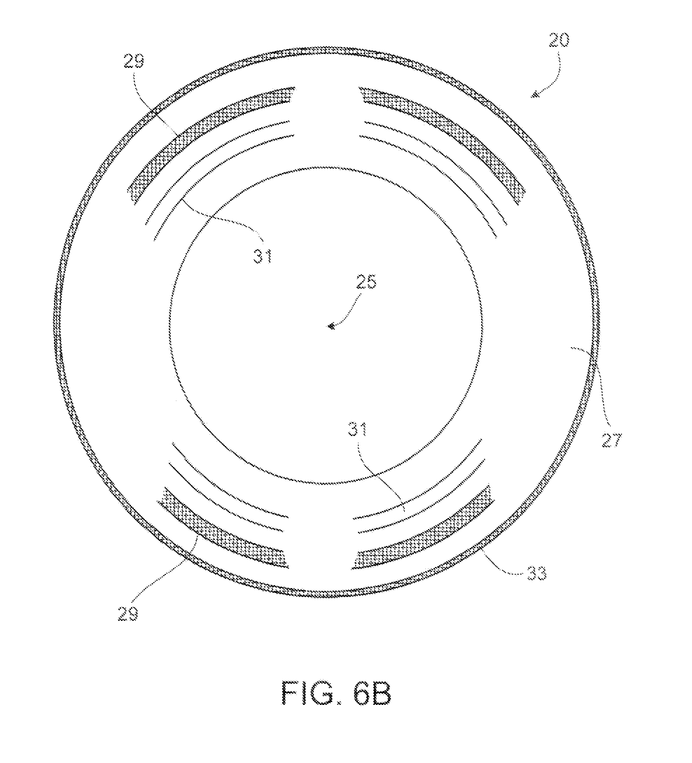

In some implementations, the stent body can have a surface with select regions having high-surface-area surface morphologies so that the stent can degrade in a controlled manner. For example, as shown in FIG. 5A, the connectors 24 of the stent 20 can include corrosion enhancing regions 33. Corrosion enhancing regions 33 can be formed by treating the desired areas of connectors 24 with a plasma immersion ion implantation process, as described further below. Inclusion of corrosion enhancing regions 33 can allow for the connectors 24 to degrade first, which can increase the flexibility of the stent along the longitudinal axis while radial opposition to the vessel wall is maintained. FIG. 5B depicts the stent after the erosion of the connectors 24, leaving the unconnected bands 22 that can still provide radial vessel opposition.

In some implementations bands 22 can include corrosion enhancing regions such as regions of high-surface-area morphologies. In some implementations bands 22 and connectors 24 can include regions of high-surface-area morphologies. In some implementations the regions of high-surface-area morphologies on bands 22 can be the same or different than the regions of high-surface-area morphologies on connectors 24. By varying the extent of high-surface-area morphologies on bands 22 and/or connectors 24, the erosion of the stent can be controlled to ensure structural integrity (e.g., radial strength and fragmentation) and stent performance (e.g., drug delivery, and cellular growth promotion) over a given period of time, e.g., greater than 1 day, greater than 7 days, greater than 14 days, greater than 30 days, between 30 and 60 days, between 60 and 360 days, or up to 720 days.

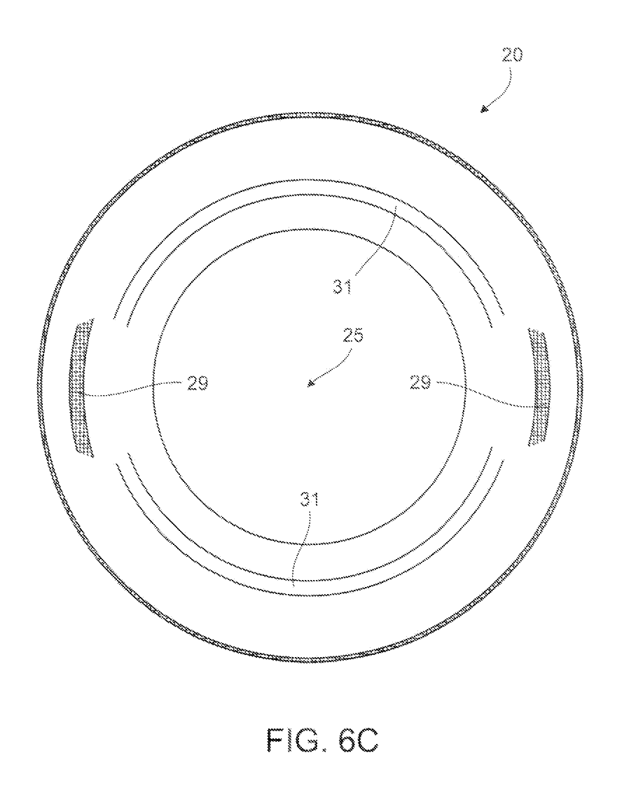

FIG. 6A depicts an exemplary cross-sectional view of a stent body 20 in accordance with further aspects of the present invention. At least a portion of the bulk 27 of the body 20 can also be treated by plasma immersion ion implantation. One or more internal portions of bulk 27 can be treated using plasma immersion ion implantation to provide layers or regions, such as modified bulk layers 29 and 31. For example, a layered structure can be created starting with a thin walled tube (e.g., a tube of approximately 50 micrometer) which in turn can be treated with a PIII treatment to create a nitride layer. A metal deposition layer can be subsequently grown over the nitride layer (e.g., growing a metal deposition layer using a sputtering process or plasma vapor deposition). Multiple differing layers can be created by alternating a PM treatment with metal deposition. Implanted ions can be diffused further into the metal by heat treatment of the PIII treated metal.

In some implementations, modified bulk layers 29 and/or 31 have different chemical compositions than the remainder the material in body 27. For example, oxygen or nitrogen ions can be implanted within a magnesium stent to create alternating layers of magnesium and magnesium oxide or nitride to provide different erosion rates. This can extend the time the magnesium stent takes to erode to a particular degree of erosion, relative to a magnesium stent without such treatment. This extension of time allows cells of the passageway in which the stent is implanted to better endothelialize around the stent, for example, before the stent erodes to a degree where it can no longer structurally maintain the patency of the passageway. The corrosion rate of magnesium can be decreased by at least a factor of 10 when the magnesium is implanted with Nitrogen. Depending on the magnesium base composition and purity, corrosion rates can range between 200 micrometers per year down to 1 micrometer per year. For example, in the case of magnesium with 9% aluminum and 1% zinc, corrosion rates between 3 and 25 micrometer per year can be obtained. The following table lists corrosion rates in micrometers per year of AZ91 as a function of impurity content (% Max impurity):

| Alloy | Cu | Ni | Fe | Mn | Corrosion Rate |
|---|---|---|---|---|---|
| AZ91D | 0.015 | 0.003 | 0.00 | 0.17 | 25 |
| AZ91X | 0.0003 | 0.003 | 0.004 | 0.17 | 12 |
| AZ91SX | 0.0024 | 0.0010 | 0.0024 | 0.17 | 5 |
| AZ91UX | 0.0010 | 0.0010 | 0.0015 | 0.17 | 3 |

Modified bulk layers 29 and 31 can have the same or different chemical compositions. For example, modified bulk layer 29 can have a first chemical composition, e.g., magnesium oxide, and modified bulk layer 31 can have a second chemical composition, e.g., magnesium nitride. Modified bulk layers 29 and 31 can be concentric and or conformal about the circumference of the stent 20, as shown in FIG. 6A. In other aspects, modified bulk layers 29 and 31 can be non-concentric as shown in FIG. 6B. In other aspects, modified bulk layers 29 and 31 can be non conformal and/or non-overlapping, as shown in FIG. 6C. In some implementations stent 20 can also include a region of enhanced surface-area modification 33 as described herein.

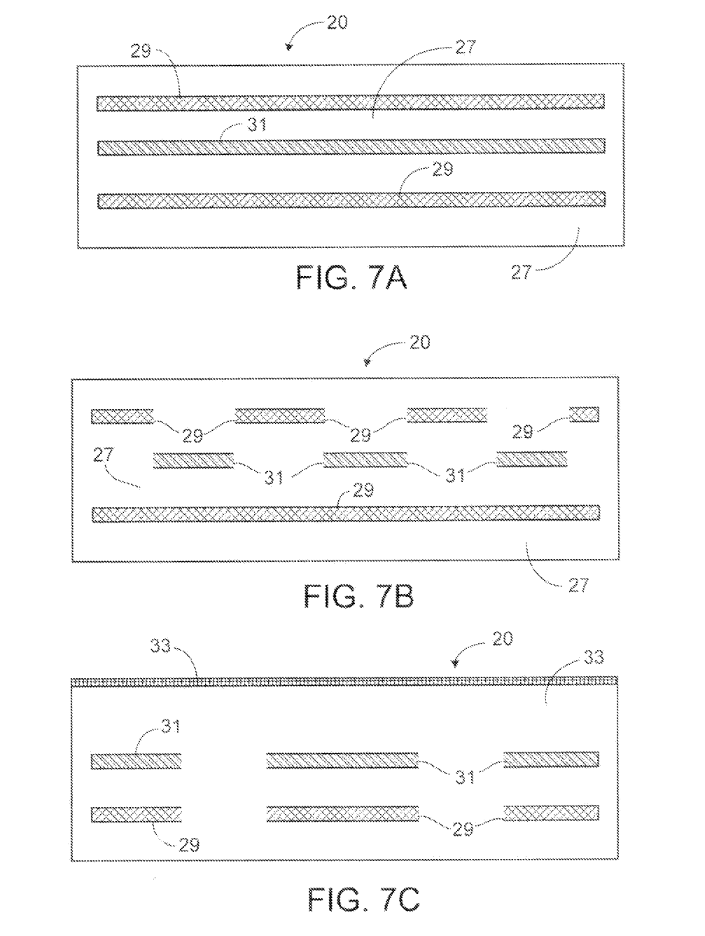

Modified bulk layers 29 and 31 can be longitudinally continuous along the longitudinal axes of the stent, as shown in FIG. 7A. Modified bulk layers 29 and 31 can be longitudinally discontinuous across stent 20, as shown in FIG. 7B. In some aspects a plurality of modified bulk layers can be included, e.g., 2 or more layers, 3 or more layers, or 4 or more layers. In some implementations stent 20 can also include a region of enhanced surface-area modification 33 as described herein, and depicted in FIG. 7C.

The bulk chemical modification and the surface morphological modification described above can be achieved in a single plasma immersion ion implantation process. During plasma immersion ion implantation, one or more charged species in a plasma, such as an oxygen and/or a nitrogen plasma, are accelerated at high velocity toward a substrate, such as a stent. Noble ions such as helium, Freon, or argon can also be used. Acceleration of the charged species, e.g., particles, of the plasma towards the substrate is driven by an electrical potential difference between the plasma and the substrate. Alternatively, one could also apply the electrical potential difference between the plasma and an electrode that is underneath the substrate such that the substrate is in a line-of-sight. Such a configuration can allow part of the substrate to be treated, while shielding other parts of the substrate. This can allow for treatment of different portions of the substrate with different energies and/or ion densities. In some embodiments, the potential difference can be greater than 10,000 volts, e.g., greater than 20,000 volts, greater than 40,000 volts, greater than 50,000 volts, greater than 60,000 volts, greater than 75,000 volts, or even greater than 100,000 volts. Upon impact with the surface of the substrate, the charged species, due to their high velocity, penetrate a distance into the substrate, mechanically and/or chemically interact with the substrate material, and form the desired surface roughness and/or porosity. Upon impact with the surface of the substrate the charged species will also cause a compressive stress in the metal layer that also influences the corrosion rate. This compressive stress can be advantageous in stent structures. For example, upon expansion of a stent, relatively large stress can occur at the intersection of two structural members, thereby causing an increased corrosion rate at these localized stress points. Pre-compensation at the intersection points by a compressive stress using PIII treatment can bring the surface stress at the intersection point to near neutral while having a compressive surface stress in the straight sections of the stent structure.

The penetration depth of the charged species can be controlled, at least in part, by the potential difference between the plasma and the substrate or electrode. Photo-lithography, stereo-lithography or similar techniques can be used to mask portions of the substrate to provide selective implantation.

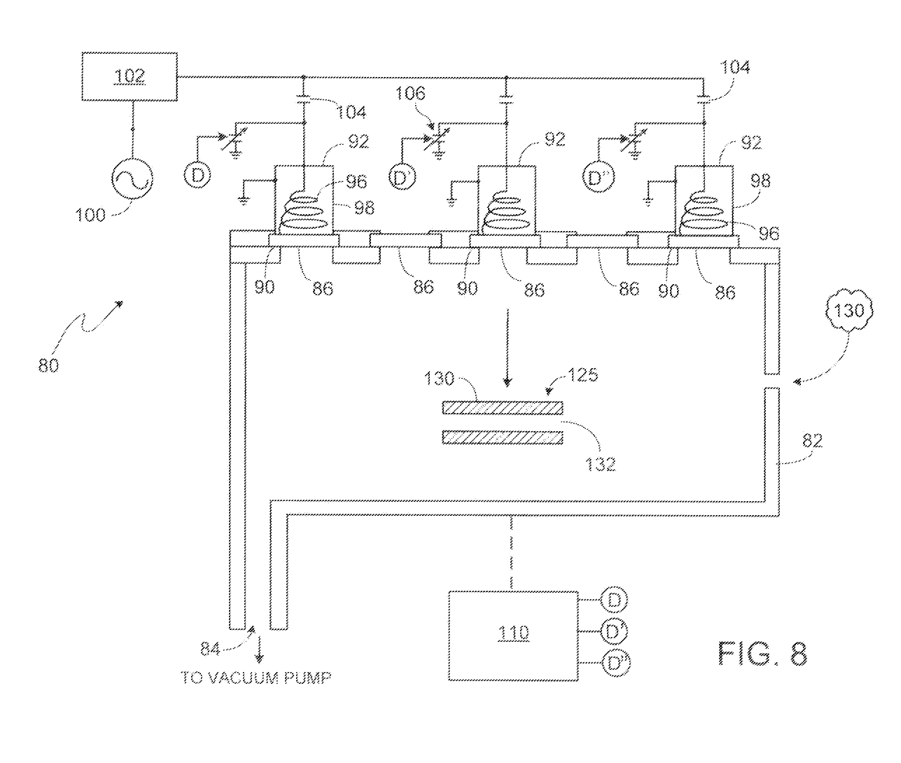

FIG. 8 shows an exemplary plasma immersion ion implantation system 80. System 80 includes a vacuum chamber 82 having a vacuum port 84 connected to a vacuum pump and a gas source 130 for delivering a gas, e.g., oxygen or nitrogen, to chamber 82 to generate a plasma. System 80 includes a series of dielectric windows 86, e.g., made of glass or quartz, sealed by o-rings 90 to maintain a vacuum in chamber 82. Removably attached to some of windows 86 are RF plasma sources 92, each source having a helical antenna 96 located within a grounded shield 98. Windows 86 without attached RF plasma sources 92 are usable, e.g., as viewing ports into chamber 82. Each antenna 96 electrically communicates with an RF generator 100 through a network 102 and a coupling capacitor 104. Each antenna 96 also electrically communicates with a tuning capacitor 106. Each tuning capacitor 106 is controlled by a signal D, D′, D″ from a controller 110. By adjusting each tuning capacitor 106, the output power from each RF antenna 96 can be adjusted to maintain homogeneity of the generated plasma.

In use, a plasma is generated in chamber 82 and accelerated to substrate 125, such as a bioerodable stent that can be made, for example, by forming a tube using a bioerodable material and laser cutting a stent pattern in the tube, or by knitting or weaving a tube from a wire or a filament made from a bioerodable material. A gas, such as oxygen, nitrogen or a silane, is introduced from gas source 130 into chamber 82, where a plasma is generated. The charged species in the generated plasma, e.g., an oxygen or nitrogen plasma, are accelerated toward exterior and/or interior portions 130, 132 of substrate 125, and thus, become implanted in substrate 125. Plasma immersion ion implantation has been described by Chu, U.S. Pat. No. 6,120,260; Brukner, Surface and Coatings Technology, 103-104, 227-230 (1998); and Kutsenko, Acta Materialia, 52, 4329-4335 (2004), the entire disclosure of each of which is herein incorporated by reference.

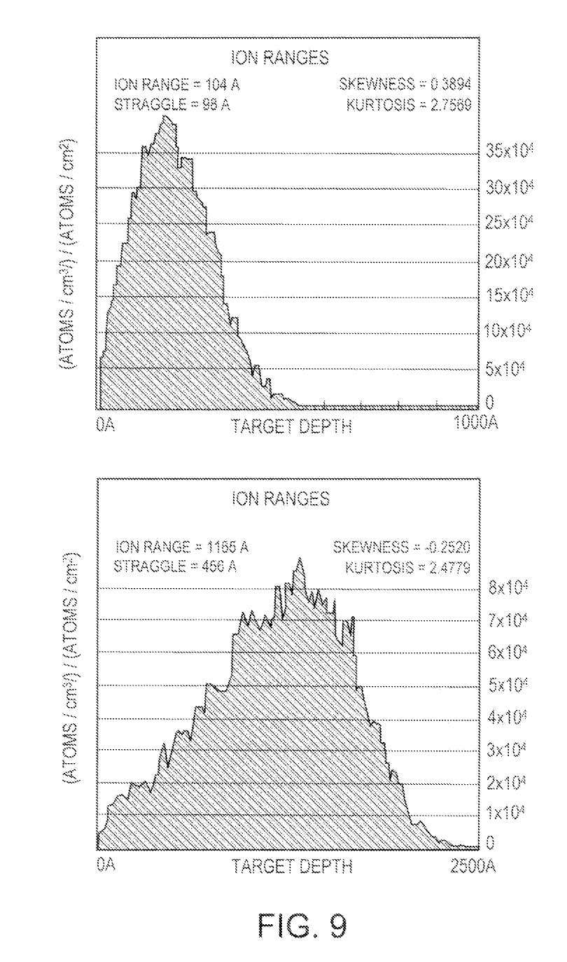

Ion penetration depth and ion concentration can be modified by changing the configuration of the plasma immersion ion implantation system. For example, when the ions have a relatively low energy, e.g., 10,000 volts or less, penetration depth is relatively shallow, compared with the situation when the ions have a relatively high energy, e.g., greater than 40,000 volts. The dose of ions applied to a surface can range from about 1×10⁴ ions/cm² to about 1×10⁹ ions/cm², preferably from about 1×10⁵ ions/cm² to about 1×10⁸ ions/cm², and can have a penetration depth of between 0 A and 2500 A, and between 0 A and 1000 A, as shown in FIG. 9. Ion penetration depth into the bulk material can also be increased by heat treatment of the material after PIII treatment. Ion penetration depth into the bulk material can also be increased using a high processing temperature during the PIII treatment.

Ion treatment of medical devices is generally described in U.S. Patent Application Publication No. US-2008-0145400-A1, filed Nov. 2, 2007 and published on Jun. 19, 2008, the contents of which are incorporated herein by reference in their entirety.

When stent 20 is bioerodable, this may change its erosion rate and hence control its service life in the body, the change in blood pH, and/or the size of the particles dispensed into the body fluid.

A stent is bioerodable if the stent or a portion thereof exhibits substantial mass or density reduction or chemical transformation, after it is introduced into a patient, e.g., a human patient. Mass reduction can occur by, e.g., dissolution of the material that forms the stent and/or fragmenting of the stent. Chemical transformation can include oxidation/reduction, hydrolysis, substitution, and/or addition reactions, or other chemical reactions of the material from which the stent or a portion thereof is made. The erosion can be the result of a chemical and/or biological interaction of the stent with the body environment, e.g., the body itself or body fluids, into which it is implanted. The erosion can also be triggered by applying a triggering influence, such as a chemical reactant or energy to the stent, e.g., to increase a reaction rate. For example, a stent or a portion thereof can be formed from an active metal, e.g., Mg or Fe or an alloy thereof, and which can erode by reaction with water, producing the corresponding metal oxide and hydrogen gas; a stent or a portion thereof can also be formed from a bioerodible polymer, or a blend of bioerodible polymers which can erode by hydrolysis with water. Fragmentation of a stent occurs as, e.g., some regions of the stent erode more rapidly than other regions. The faster eroding regions become weakened by more quickly eroding through the body of the endoprosthesis and fragment from the slower eroding regions.

Preferably, the erosion occurs to a desirable extent in a time frame that can provide a therapeutic benefit. For example, the stent may exhibit substantial mass reduction after a period of time when a function of the stent, such as support of the lumen wall or drug delivery, is no longer needed or desirable. In certain applications, stents exhibit a mass reduction of about 10 percent or more, e.g. about 50 percent or more, after a period of implantation of about one day or more, about 60 days or more, about 180 days or more, about 600 days or more, or about 1000 days or less.

Erosion rates can be adjusted to allow a stent to erode in a desired sequence. For example, regions can be treated to increase erosion rates by enhancing their chemical reactivity. Alternatively, regions can be treated to reduce erosion rates, e.g., by using coatings. Erosion rates can be measured with a test stent suspended in a stream of Ringer's solution flowing at a rate of 0.2 m/second. During testing, all surfaces of the test stent can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter.

After the plasma immersion ion implantation treatment, a coating can be deposited over the treated surface of stent 20 to provide a desired function. Examples of such coatings include a tie layer, a biocompatible outer coating, a radiopaque metal or alloy, and/or a drug-eluting layer. The surface treatment may improve the adhesion between the coating and the stent surface.

The treated surface of stent 20 can be incorporated with at least one releasable therapeutic agent, drug, or pharmaceutically active compound to inhibit restenosis, such as paclitaxel, or to treat and/or inhibit pain, encrustation of the stent or sclerosing or necrosing of a treated lumen. The therapeutic agent can be a genetic therapeutic agent, a non-genetic therapeutic agent, or cells. The therapeutic agent can also be nonionic, or anionic and/or cationic in nature. Examples of suitable therapeutic agents, drugs, or pharmaceutically active compounds include anti-thrombogenic agents, antioxidants, anti-inflammatory agents, anesthetic agents, anti-coagulants, and antibiotics, as described in U.S. Pat. No. 5,674,242; U.S. Ser. No. 09/895,415, filed Jul. 2, 2001; U.S. Ser. No. 11/111,509, filed Apr. 21, 2005; and U.S. Ser. No. 10/232,265, filed Aug. 30, 2002, the entire disclosure of each of which is herein incorporated by reference. Representative conventional approaches disperse the therapeutic agent, drug, or a pharmaceutically active compound in a polymeric coating carried by a stent. In the present invention, the therapeutic agent, drug, or a pharmaceutically active compound can be directly incorporated into the pores generated by plasma immersion ion implantation treatment on the surface of a stent, thereby eliminating the use of extra coatings.

Stent 20 can have any desired shape and size (e.g., superficial femoral artery stents, coronary stents, aortic stents, peripheral vascular stents, gastrointestinal stents, urology stents, and neurology stents). Depending on the application, stent 20 can have an expanded diameter of about 1 mm to about 46 mm. For example, a coronary stent can have an expanded diameter of about 2 mm to about 6 mm; a peripheral stent can have an expanded diameter of about 5 mm to about 24 mm; a gastrointestinal and/or urology stent can have an expanded diameter of about 6 mm to about 30 mm; a neurology stent can have an expanded diameter of about 1 mm to about 12 mm; and an abdominal aortic aneurysm stent and a thoracic aortic aneurysm stent can have an expanded diameter of about 20 mm to about 46 mm. Stent 20 can be self-expandable, balloon-expandable, or a combination of self-expandable and balloon-expandable (e.g., as described in U.S. Pat. No. 5,366,504). Stent 20 can have any suitable transverse cross-section, including circular and non-circular (e.g., polygonal such as square, hexagonal or octagonal).

Stent 20 can be implemented using a catheter delivery system. Catheter systems are described in, for example, Wang U.S. Pat. No. 5,195,969; Hamlin U.S. Pat. No. 5,270,086; and Raeder-Devens, U.S. Pat. No. 6,726,712, the entire disclosure of each of which is herein incorporated by reference. Commercial examples of stents and stent delivery systems include Radius®, Symbiot® or Sentinol® system, available from Boston Scientific Scimed, Maple Grove, Minn.

Stents 20 can be a part of a covered stent or a stent-graft. For example, stent 20 can include and/or be attached to a biocompatible, non-porous or semi-porous polymer matrix made of polytetrafluoroethylene (PTFE), expanded PTFE, polyethylene, urethane, or polypropylene.

In addition to vascular lumens, stent 20 can be configured for non-vascular lumens. For example, it can be configured for use in the esophagus or the prostate. Other lumens include biliary lumens, hepatic lumens, pancreatic lumens, uretheral lumens and ureteral lumens.

The present invention has now been described with reference to several embodiments thereof. The foregoing detailed description has been given for clarity of understanding only. No unnecessary limitations are to be understood therefrom. All patents and patent applications cited herein are hereby incorporated by reference. It will be apparent to those skilled in the art that many changes can be made in the embodiments described without departing from the scope of the invention. Thus, the scope of the present invention should not be limited to the exact details and structures described herein, but rather by the structures described by the language of the claims, and the equivalents of those structures.

What is claimed is:

1. An endoprosthesis comprising a member, the member further comprising a bioerodable portion, wherein at least a portion of a surface of the bioerodable portion is treated by plasma immersion ion implantation.

2. The endoprosthesis of claim 1, wherein the portion of the surface treated by plasma immersion ion implantation includes a region of enhanced surface-area morphology.

3. The endoprosthesis of claim 1, wherein the member is a connector of a stent

4. The endoprosthesis of claim 1, wherein the member is a circumferential band of a stent.

5. The endoprosthesis of claim 1, wherein the surface is an abluminal surface.

6. The endoprosthesis of claim 1, wherein the surface is a luminal surface.

7. The endoprosthesis of claim 1, wherein the surface is a sidewall surface.

8. The endoprosthesis of claim 1, wherein a coating is deposited over the treated surface.

9. The endoprosthesis of claim 8, wherein the coating comprises a tie layer, a biocompatible outer coating, a radiopaque metal or alloy, a drug-eluting layer, or a combination thereof.

10. The endoprosthesis of claim 1, wherein at least one releasable therapeutic agent, drug, or pharmaceutically active compound is incorporated into the treated surface.

11. The endoprosthesis of claim 10, wherein the releasable therapeutic agent, drug, or pharmaceutically active compound comprises anti-thrombogenic agent, antioxidant, anti-inflammatory agent, anesthetic agent, anti-coagulant, antibiotic, or combination thereof.

12. The endoprosthesis of claim 1 wherein the bioerodable material comprises a metal.

13. The endoprosthesis of claim 12, wherein the metal comprises iron, magnesium, or an alloy thereof.

14. The endoprosthesis of claim 1, wherein the bioerodable material comprises a polymer.

15. The endoprosthesis of claim 14, wherein the polymer comprises polydioxanone, polycaprolactone, polygluconate, polylactic acid-polyethylene oxide copolymer, modified cellulose, collagen, poly(hydroxybutyrate), polyanhydride, polyphosphoester, poly(amino acid), poly-L-lactide, poly-D-lactide, polyglycolide, poly(alpha-hydroxy acid), or combination thereof.

16. The endoprosthesis of claim 1, wherein the member comprises a non-bioerodable material.

17. An endoprosthesis comprising a member, the member further comprising a bioderodable, wherein at least a portion of the bulk of the member is treated by plasma immersion ion implantation.

18. The endoprosthesis of claim 17, wherein the portion treated by plasma immersion ion implantation is at least a portion of the bioerodable portion.

19. The endoprosthesis of claim 17, wherein the portion treated by plasma immersion ion implantation is disposed in the bulk of the member at a depth between 0 A and 2500 A.

20. The endoprosthesis of claim 17, wherein the portion treated by plasma immersion ion implantation includes a first portion adjacent a second untreated portion.

21. The endoprosthesis of claim 20, wherein the portion treated by plasma immersion ion implantation includes a first portion and a second portion, the first and second portions having different chemical compositions than the bulk material of the member.

22. The endoprosthesis of claim 21, wherein the first portion is at a first depth within the bulk of the member and the second portion is at a second depth within the bulk of the member.

23. The endoprosthesis of claim 21, wherein the first portion and the second portion at least partially overlap.

24. The endoprosthesis of claim 21 wherein the first portion and the second portion are at least partially concentric about the circumference of the member.

25. The endoprosthesis of claim 21, wherein the first portion and the second portion at least partially extend along the longitudinal axis of the endoprosthesis.

26. The endoprosthesis of claim 17 wherein the member is a connector of a stent.

27. The endoprosthesis of claim 17 wherein the member is a circumferential band of a stent.

28. The endoprosthesis of claim 17, wherein a plurality of portions of the bulk of the member are treated by plasma immersion ion implantation to different chemical compositions.

29. The endoprosthesis of claim 17, wherein the bioerodable material comprises a metal.

30. The endoprosthesis of claim 29, wherein the metal comprises iron, magnesium, or an alloy thereof.

31. The endoprosthesis of claim 17, wherein the bioerodable material comprises a polymer.

32. The endoprosthesis of claim 31, wherein the polymer comprises polydioxanone, polycaprolactone, polygluconate, polylactic acid-polyethylene oxide copolymer, modified cellulose, collagen, poly(hydroxybutyrate), polyanhydride, polyphosphoester, poly(amino acid), poly-L-lactide, poly-D-lactide, polyglycolide, poly(alpha-hydroxy acid), or combination thereof.

33. The endoprosthesis of claim 17, wherein the member comprises a non-bioerodable material.

34. The endoprosthesis of claim 33, wherein the non-bioerodable material comprises stainless steel, platinum enhanced stainless steel, cobalt-chromium alloy, nickel titanium alloy, or combination thereof.

35. An endoprosthesis comprising a member, the member further comprising:
a biodegradable portion, wherein at least a portion of a surface of the biodegradable portion is treated by plasma immersion ion implantation; and
wherein at least a portion of the bulk of the member is treated by plasma immersion ion implantation.

36. A method of forming an endoprosthesis comprising:
treating a portion of the surface of an endoprosthesis with plasma immersion ion implantation; and
treating a portion of the bulk material of a portion of the endoprosthesis with plasma immersion ion implantation.

37. The method of claim 36 wherein the portion of the surface treated comprises a bioerodable portion of the endoprosthesis.

38. The method of claim 36 wherein the portion of the surface treated comprises an enhanced surface-area morphology.

38. The method of claim 36 wherein the portion of the bulk material treated comprises a bioerodable portion of the endoprosthesis.

39. The method of claim 36 wherein the portion of the bulk material treated comprises a first treated portion having a different chemical composition than the bulk material.

40. The method of claim 36 wherein the portion of the bulk material treated is treated by plasma immersion ion implantation at a depth between 0 A and 2500 A.
